SEQUENCE LISTING (2 sequences)

SEQ ID NO: 1 - DNA, 12 bp, Artificial
  Linking nucleotide
gccgccgccatg

SEQ ID NO: 2 - protein, 4 aa, Artificial
  Linking peptide
AAAM

(19) United States
(12) Patent Application Publication
(10) Pub. No.: US 2007/0042485 A1
(43) Pub. Date: Feb. 22, 2007

(54) Modified TRAP protein for producing nano-scale electrical devices, and a method for producing such a protein

(75) Inventors: Jonathan Heddle, Yokohama-shi (JP); Jeremy R.H. Tame, Yokohama-shi (JP)

Correspondence Address: FOLEY AND LARDNER LLP;SUITE 500, 3000 K STREET NW, WASHINGTON, DC, 20007, US

(73) Assignees: JAPAN SCIENCE AND TECHNOLOGY AGENCY; YOKOHAMA CITY UNIVERSITY

(21) Appl. No.: 11/453,232

(22) Filed: Jun. 15, 2006

(30) Foreign Application Priority Data
Aug. 17, 2005 (JP) 2005-236771

(51) Int. Cl. H01L 51/00; G06F 19/00; C12P 21/06; C12N 9/22; C12N 1/21

(52) U.S. Cl. 435/199; 438/1; 435/69.1; 435/320.1; 435/252.31; 977/702; 702/19

(57) ABSTRACT

The present invention provides a modified TRAP protein formed from a plurality of genetically engineered fusion molecules of TRAP monomers, wherein said fusion molecule consists of two, three or four wild type TRAP monomer equivalents.

### BACKGROUND OF THE INVENTION

1. Field of the Invention

This invention relates to modified TRAP proteins for use as templates and/or reaction vessels, and a method for producing such proteins. Specifically, the invention relates to modification of bacterial TRAP proteins to change their properties such that they can form ring like structures of useful symmetry and tube structures the cavities of which could be used as a reaction vessel for the production of nanoparticles (such as quantum dots) and nanowires for use in electronic devices.

2. Description of the Related Art

The continuing miniaturization of semiconductor devices is a desirable trend that allows faster, more powerful computers and the continued advancement of technology as a whole. Current methods used in constructing nano-scale electronic devices typically rely on lithography in which components are etched from silicon wafers using electromagnetic radiation of a short wavelength. In this method, layers of silicon dioxide, polysilicon and photoresist are layered over a positively doped silicon wafer surface. A photomask is placed over the photoresist. The surface is exposed to electromagnetic radiation with the photomask shielding parts of the surface according to a specific pattern. The photoresist modified by exposure to the radiation and either modified or unmodified photoresist can then be removed.

Problems with this technology include excessive power consumption and heat generation in the final product, ability to produce only 2-dimensional components, fundamental limitations in the size of the components produced due to the wavelength of light used, limitations in the dimensions of the photomask used and cost intensive manufacturing. In order to continue the trends in miniaturization, new methods will have to be used to build smaller components and eventually, the problems of heat production will have to be addressed.

On the other hand, there has been also a report of using a bacterial protein to make gold nanodots (McMillan et al., Nat Mater 1, 247-252, 2002).

### SUMMARY OF THE INVENTION

Under the circumstances described above, a new method of producing electric devices of small scale, such as in the 10s of nanometer size range, that circumvents some or all of the above mentioned problems is desired.

Specifically, a new method for producing such small scale conducting and/or semiconducting devices, with lower costs and lower heat production than the conventional methods, is desired.

Also desired is to produce such small scale conducting and/or semiconducting devices, whose dimensions can be modified.

Also desired is a material which can be used to produce such small scale devices and which can be modified to make further useful templates for producing such devices.

In view of the above, the present invention provides the following:

(1) A modified TRAP protein formed from a plurality of genetically engineered fusion molecules of TRAP monomers, wherein said fusion molecule consists of two, three four, or more wild type TRAP monomer equivalents.

(2) The crystal of the above item (1), wherein said fusion molecule consists of three or four wild type TRAP monomer equivalents.

(3) The protein of the above item (1) or (2), which forms a ring containing the equivalent of 12 wild type TRAP monomers.

(4) The protein of any one of the above items (1) to (3), wherein the wild type TRAP monomers in the fusion molecules are linked by a linking peptide.

(4a) The protein of the above item (4), wherein the linking peptide consists of Ala-Ala-Ala-Met.

(5) The protein of any one of the above items (1) to (4a), wherein the gene of said TRAP protein is derived from Bacillus stearothermophilus.

(6) A nanotube formed from a multiple copies of the modified TRAP protein as defined by any one of the above items (1) to (5), wherein the modified TRAP proteins stack in an aligned manner to form a tube.

(7) A crystal of the protein of the above item (2), which has lattice constants: a=110±2 Å, b=110±2 Å, and c=36±2 Å, and is crystallized in space group P42₁2.

(8) A crystal of the nanotube of the above item (6), wherein the modified TRAP proteins stack in an aligned manner to form close packed, linear nanotubes.

(9) A biomineralization vessel comprising the protein of any one of the above items (1) to (5), or the nanotube of claim 6.

(10) A quantum dot comprising the protein of any one of the above items (1) to (5) and metal.

(10a) The quantum dot of the above item (10), wherein the metal is entrapped in the central cavity of the ring of said modified TRAP protein.

(10b) The quantum dot of the above item (10) or (10a), wherein the metal is gold.

(11) A nanowire comprising the nanotube of the above item (6) and metals.

(11a) The nanowire of the above item (11), wherein the metal is entrapped in the central cavity of the ring of said modified TRAP protein.

(11b) The nanowire of the above item (11) or (11a), wherein the metal is gold.

(12) A method for producing a modified TRAP protein formed from a plurality of genetically engineered fusion molecules of TRAP monomers, or a nanotube formed from multiple copies of said modified TRAP protein, which method comprising:

preparing a gene construct comprising a multiple TRAP monomer genes;

expressing the gene construct in culture cells to produce a genetically engineered fusion molecules of TRAP monomers; and

purifying the expressed fusion molecules.

(12a) The method of the above item (12), wherein the culture cells are E. coli cells.

(13) The method of the above item (12), wherein the gene of said TRAP protein is derived from Bacillus stearothermophilus.

(13a) The method of the above item (12), wherein the fusion molecules self-assemble into a 12-membered TRAP ring.

(14) A method for producing ultra-thin semiconductor wires or switches comprising using the nanotube of the above item (6) as masks for photolithography.

The invention allows the production of quantum dots (see FIG. 6) and nanowires that can be used to make transistors and electronic components of considerably smaller dimensions than is currently possible using conventional techniques.

The diameter of the central hole of the modified TRAP ring of the present invention can be as small as about 30 Å. The small diameter of the TRAP ring of the present invention is smaller than the component size currently produceable by the semiconductor industry. Smaller dimensions should lead to faster computing devices as the distance that electrons need to travel is reduced and also the number of components that can fit into a given area is increased. Having such a small size should allow such components to operate using fewer electrons. If the size and spacing between components is sufficiently small it could allow operation using only one electron, thus reducing heat output per component considerably.

In addition the ability of proteins to self-assemble should offer distinct cost advantages over current manufacturing techniques.

Further, the modified TRAP protein of the present invention provides a cage protein for biomineralization whose dimensions can be modified and whose symmetry can be altered to better produce tightly and regularly packed quantum dots on a surface. Such dots could be used as photomasks of smaller dimension than those currently used or, directly as components of an electron-transfer device.

Furthermore, if the quantum dots are placed close enough to each other they may be utilized as single electron transistors, a vital component of proposed quantum computing devices.

### BRIEF DESCRIPTION OF THE SEVERAL VIEWS OF THE DRAWINGS

FIG. 1 shows a schematic diagram showing construction of a multiple-TRAP gene.

FIG. 2 shows a TEM image of TRAP3 deposited on a carbon coated copper mesh. A large number of ring-like structures can be seen. The red square is enlarged in the inset, showing a single TRAP ring (highlighted in green circle) measuring approximately 9.1 nm in diameter.

FIG. 3 shows crystal structures showing the packing of proteins in crystals of A. wild type TRAP from Bacillus stearothermophilus, B. wild type TRAP from Bacillus stearothermophilus with RNA bound to one ring, C. wild type TRAP from Bacillus subtilis (20) and D. TRAP3 . Structures are shown “face on” on the left and from the “side” on the right. The structure for TRAP4 is virtually identical to TRAP3.

FIG. 4 shows a schematic diagram showing the connection between monomer equivalents in A, the TRAP3 protein and B, the TRAP4 protein.

FIG. 5 shows a schematic showing the crystal packing of TRAP molecules. A. wild type TRAP from Bacillus stearothermophilus B. wild type TRAP from Bacillus stearothermophilus with RNA bound to one ring C. wild type TRAP from Bacillus subtilis and D. TRAP3 and TRAP4. Only for TRAP3 and TRAP4 do the neighbouring rings align holes and stack in a unique orientation.

FIG. 6 shows a schematic showing the proposed interaction of a modified TRAP ring with a gold nanoparticle (i). The ring can bind the gold in the central cavity to form a quantum dot (ii).

### DETAILED DESCRIPTION OF THE INVENTION

1. A Modified TRAP Protein

In one aspect of the present invention, there is provided a modified TRAP protein formed from a plurality of genetically engineered fusion molecules of TRAP monomers. The fusion molecule consists of at least two wild type TRAP monomer equivalents.

The term “TRAP” as used in the present specification refers to a bacterial trp RNA-binding attenuation protein (TRAP). Bacteria from which the TRAP protein can be derived include Bacillus stearothermophilus, Bacillus subtilis and Bacillus pumilis. Among others, Bacillus stearothermophilus is the most preferable origin of the TRAP gene or protein for use in the present invention. Wild type TRAP protein typically consists of 11 monomer subunits. Sequence information on Bacillus stearothermophilus TRAP protein and the gene encoding thereof can be obtained from publicly available sequence databases such as SwissProt with an accession number of Q9X6J6. Sequence information on other bacterial TRAP proteins is also available from publicly available sequence databases. Structural information on natural TRAP proteins is available through the Protein DataBank with accession codes 1QAW, 1C9S, 1GTF, 1UTD, 1UTF and 1UTV.

The fusion molecules of TRAP monomers can be produced using conventional techniques of genetic engineering (e.g., Sambrook and Russell, Molecular Cloning; A Laboratory Manual, Third Edition, 2001 by Cold Spring harbor Laboratory Press, Cold Spring Harbor, N.Y.). The fusion molecule of TRAP monomers used in the present invention consists of at least two wild type TRAP monomers. In one embodiment of the present invention, the fusion molecule consists of 3 or 4 wild type TRAP monomer equivalents. The fusion molecules self assemble into a 12-membered TRAP ring consisting of 3 or 4 fusion molecules such that the ring consists of the equivalent of 12 wild type monomer subunits rather than the conventional 11-membered ring found naturally. Our X-ray crystal structures of TRAP3 and TRAP4 show the structure of this 12mer ring in atomic detail.

This 12-membered ring has the advantages of symmetry favourable to forming regular, tightly packed arrays in 2 dimensions, and, because it is made by 3 or 4 fusion proteins, it can be non-homogeneously mutated. If different sizes of ring cavity are required, this may be possible by fusion of different numbers (such as 5, 6, 7, or 8) of TRAP monomers.

The TRAP fusion molecules of the present invention consisting of 3 or 4 wild type TRAP monomer equivalents provide a cage protein for biomineralization with suitable symmetry to produce tightly and regularly packed quantum dots on a surface. Such quantum dots can be made by mineralizing conducting material in the central core of the TRAP ring. The quantum dots could be used as photomasks of smaller dimension than those currently used or, directly as components of an electron-transfer device. The conducting materials that can be used for such purpose include gold, silver, platinum, iron, and cadmium. Among others, gold is most preferable.

In another aspect of the present invention, the present invention provides a nanotube, which is formed from multiple copies of the modified TRAP protein described above. The present invention utilizes the modified TRAP proteins that stack to form long tubes. The tube will be utilized to make nanowires by mineralizing conducting materials in its central core. The conducting materials that can be used for such purpose include gold, silver, platinum, iron, and cadmium. Among others, gold is most preferable. Thus the present invention will allow the production of nanowires of small (approximately 10-20 nm) diameter.

2. A Method for Producing a Modified TRAP Protein

In another aspect of the present invention, there is provided a method for producing modified TRAP proteins formed from a plurality of genetically engineered fusion molecules of TRAP monomers, or a nanotube formed from a multiple copies of said modified TRAP protein. This method comprises preparing a gene construct comprising multiple TRAP monomer genes; expressing the gene construct in culture cells to produce genetically engineered fusion molecules of TRAP monomers; and purifying the expressed fusion molecules.

The gene construct can be prepared using the gene encoding bacterial TRAP, preferably Bacillus stearothermophilus TRAP according essentially to a procedure well known in the field of genetic engineering. Other bacterial TRAP genes, which can be used in the present invention, include those of Bacillus subtilis and Bacillus pumilis. For the culture cells, E. coli cells are preferable. Other culture cells, which can be used for the purpose of the present invention, include yeast. In vitro systems may also be used.

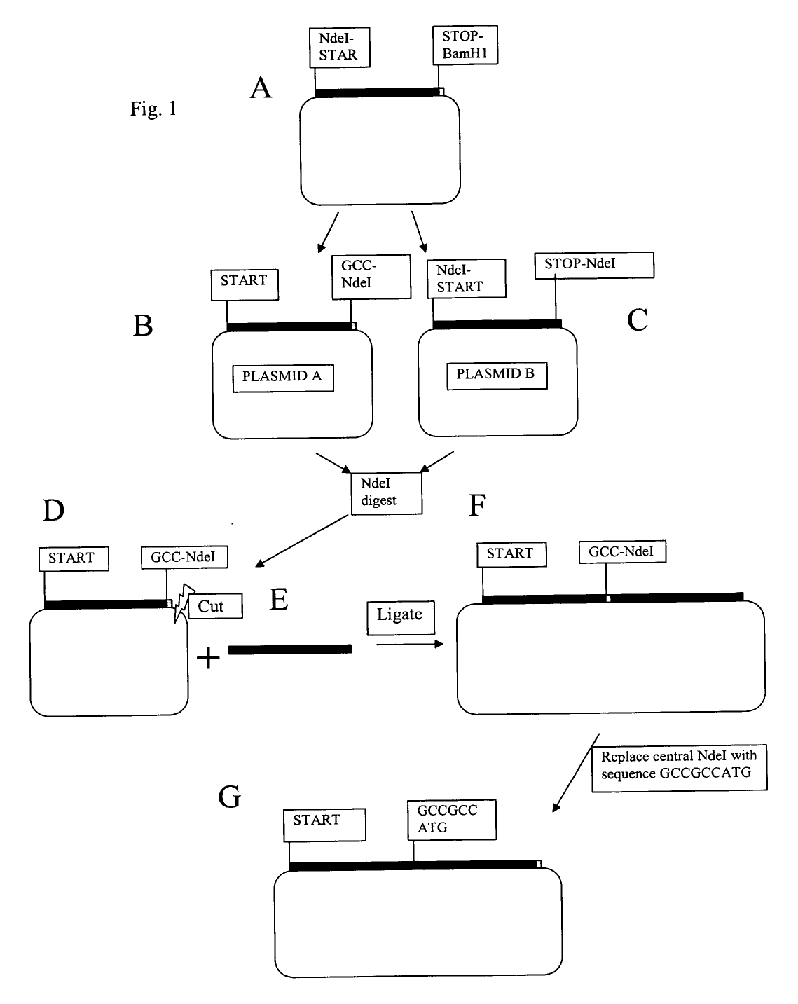

One example of preparation of a gene construct is described with reference to FIG. 1. FIG. 1 shows a schematic diagram of constuction of a multiple TRAP gene A) in the initial plasmid, the gene encoding wild type Bacillus stearothermophilus TRAP monomer was cloned into the NdeI and BamHI sites of expression plasmid pET21b. This plasmid was mutated to form, B) plasmid A in which the N-terminal NdeI site is removed, the STOP codon is replaced with sequence GCC and the C-terminal BamHI site is replaced with an NdeI site and C) in which the C-terminal BamHI site is replaced with an NdeI site. Both plasmids are digested with NdeI to yield D) a plasmid containing one copy of the TRAP gene linearized at the C-terminus and E) a linear piece of DNA encoding the TRAP gene with digested NdeI sites at both ends. Purification and ligation of these two products yields F) a plasmid containing two copies of the TRAP gene with a central NdeI site. The central NdeI site is replaced with the linking sequence GCCGCCATG to yield G) a plasmid encoding a new gene consisting of a fusion of two TRAP monomer genes. Further mutation of this plasmid can return the N and C-termini sequences to those in A) and the process can then be repeated to add more copies of the TRAP gene.

Purification of the expressed fusion molecules can be typically performed using an ion-exchange column, such as Q-sepharose (Pharmacia), with, for example, ascending NaCl gradient from, for example, 0 mM to 1 M NaCl. The expressed molecules may be further purified, as necessary, using, for example an affinity column such as a heparin sepharose column (Pharmacia), and/or an gel filtration column such as HiLoad superdex 200 (Pharmacia).

Crystallization of the modified TRAP protein of the present invention can be performed according to a conventional crystallization method well known in the art. Such crystallization method includes; sitting drop vapor diffusion method, hanging drop vapor diffusion method, sandwich drop method, batch method, microbatch method, under oil method, microdialysis method, and free interface diffusion method. For the purpose of the present invention, vapor diffusion method is preferable, and hanging drop vapor diffusion method is most preferable.

Briefly, in the hanging drop method, a small (0.1 to 40 microliters) droplet of the sample mixed with crystallization reagent is placed on a siliconized glass cover slide inverted over the reservoir in vapor equilibration with the reagent. The initial reagent concentration in the droplet is less than that in the reservoir. Over time the reservoir will pull water from the droplet in a vapor phase such that an equilibrium will exist between the drop and the reservoir. During this equilibration process the sample is also concentrated, increasing the relative supersaturation of the sample in the drop.

The concentration of the protein in the sample droplet can be about 1 to 20 mg/ml. The sample droplet may contain a buffering agent such as CAPS or Tris at a concentration between 50 mM and 100 mM in order to keep the pH of the sample droplet at about 9 to 10. The sample droplet may also contain a precipitating agent such as polyethylene glycol (PEG) with various molecular weights. Among others, PEG 300 is most preferable for the present invention. Crystallization is normally performed at 4 to 25° C., and a crystal can be obtained after, for example, several days or a few weeks.

In another aspect of the present invention, there is provided a method for production of a nano-ring or a nanotube, within a crystal, using the same modified bacterial protein so as to provide templates for producing quantum dots or nanowires.

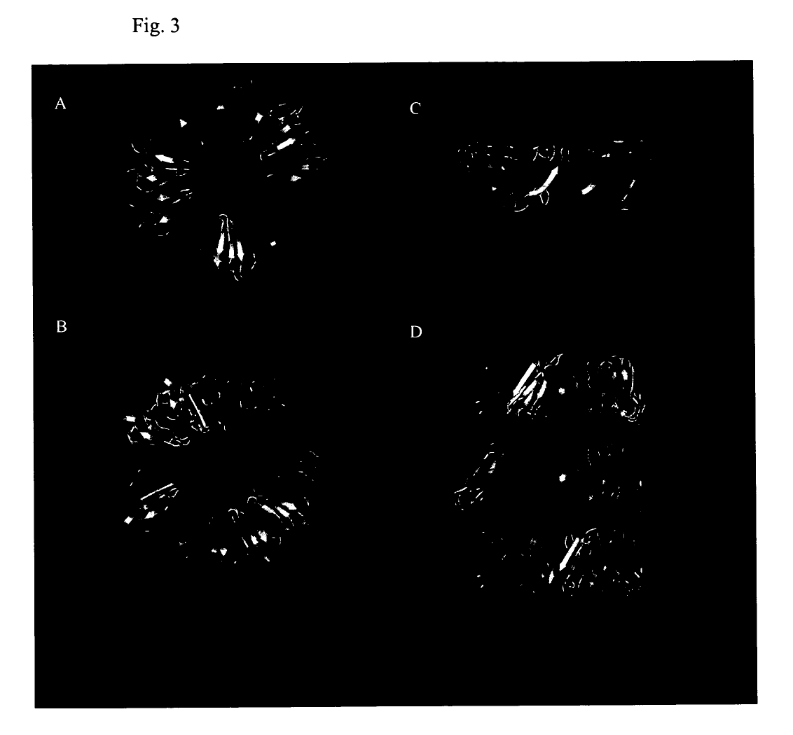

In one embodiment of the present invention, the structures of TRAP3 and TRAP4 (see FIG. 3) show almost identical tertiary structure to the wild-type TRAP monomer. The rings formed by TRAP3 and TRAP4 are essentially identical in size and have a central hole 31.1 Å in diameter (between opposing Ser7 Cαs), the height of the ring is 31.8 Å (between Lys40Cα and Asp29 Cα) and the overall diameter is 81.0 Å (between opposing Gly18 Cαs) compared to 27.7 Å, 32.1 Å and 77.0 Å respectively in the wild-type structure. It would be understood that such dimensions of the ring structure might vary depending on the number of monomers contained therein.

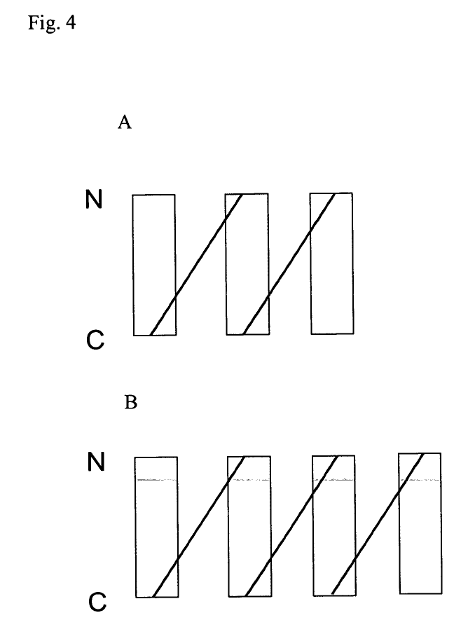

The monomers of TRAP3 and TRAP4 are constructed from 3 and 4 wild type monomer equivalents respectively, and the domains are linked by linking peptide strands as shown in FIG. 4. In the present specification, the term “monomer equivalents” is used to mean that the fusion molecules not only contain polypeptide corresponding to the wild type monomer but also the linking peptide.

FIG. 4 shows a schematic diagram showing the probable connection between monomer equivalents within the fusion protein. In the figure, the monomer equivalents are shown as blue blocks. N and C-termini equivalents are marked as N and C respectively. In the wild type monomer protein, the N and C-terminal residues are likely to lie at opposite faces of the ring with the N-terminal residue pointing into the cavity and the C-terminal residue lying on the outer surface of the ring. The distance between the last visible N and C-terminal residues in the wild type structure (Ser 5 and Lys 73 respectively) is 37.8 Å, the direction of the amino acid chains suggesting that the actual distance between residue 1 and C-terminal residue 74 is greater. In the constructed protein, a loop of amino acids (shown by the red line, not visible in the structure) must connect the monomer equivalents as shown.

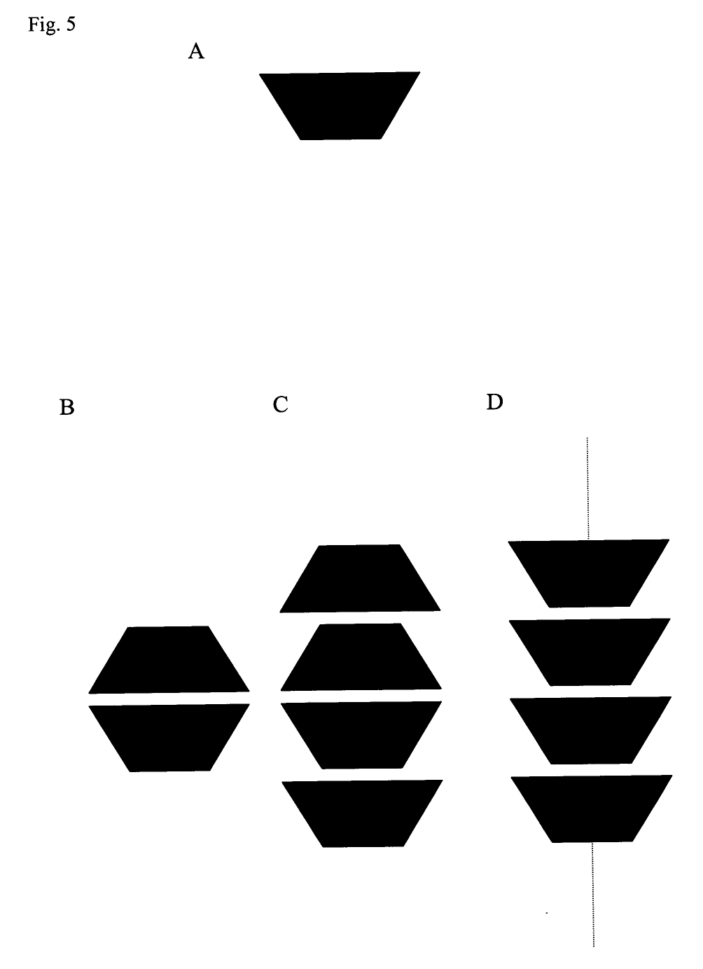

FIG. 5 shows a schematic diagram showing the packing of TRAP molecules in the crystal for A) wild type TRAP from Bacillus stearothermophilus; B) wild type TRAP from Bacillus stearothermophilus with RNA bound to one ring; C) wild type TRAP from Bacillus subtilis and D) TRAP3 and TRAP4 of the present invention. In the figure, the RNA is not shown in B for clarity. In A, the TRAP ring has few contacts with other rings and does not align to form a tubular structure. In B and C, tubular structures do form but the interactions between TRAP rings are such that the tubes are capped after 2 and 4 repeats respectively and do not for longer tubes in the crystal. For TRAP3 and TRAP4 however, the interactions between the rings mean that the tube formed stretches indefinitely (represented by dotted line).

In the crystal structure, the modified TRAP3 and TRAP4 proteins stack in an aligned manner to form an essentially infinite tube (see FIG. 5D). In other structures of TRAP solved, packing does not result in the formation of a continuous tube (see FIG. 5B and C).

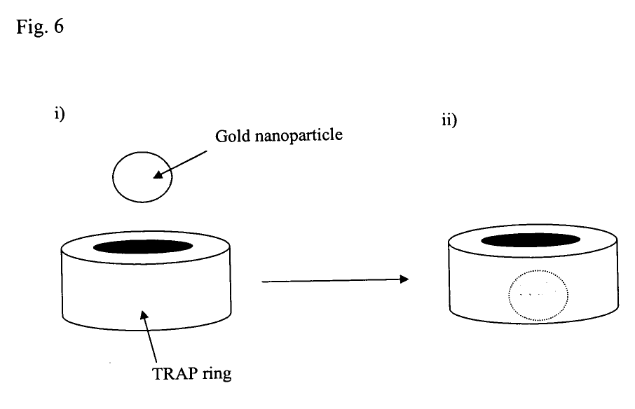

Thus, in a typical example of the modified TRAP protein of the present invention, the fundamental subunits of which are made from either 3 (in the case of TRAP3) or 4 (in the case of TRAP4) fused monomer proteins. These subunits self assemble into TRAP rings consisting of the equivalent of 12 wild type monomer subunits. We have made tubes within crystals using this protein. The present inventors have also arrayed the ring proteins on a carbon grid surface. These tubes and rings can be used to biomineralize metals such as gold to form quantum dots and nanowires (see FIG. 6).

It should be understood that the TRAP gene for use in any aspects of the present invention may include a variant of any wild type TRAP gene, which encodes a variant TRAP protein which consists of amino acid sequence having one or more (for example, several (e.g., 1-20, 1-15, 1-10, 1-6, or 1-3)) amino acid substitutions, additions, and/or deletions in the original wild type TRAP as long as a modified TRAP ring or a nanotube comprising thereof according to the present invention can be produced.

In the following Examples, the present invention is described more specifically. However, it should be understood that the present invention would not be limited to the following Examples.

### EXAMPLES

Construction of a Multiple-TRAP Gene

A multiple-TRAP gene s constructed as shown in FIG. 1. The gene encoding for Bacillus stearothermophilus TRAP was sequence-optimized for expression in E. coli and cloned into pET 28b (Novagen) at the NdeI and BamHI restriction enzyme sites by Genscript Corporation (NJ, USA). In order to insert a second copy of the TRAP gene, two copies of the plasmid carrying the TRAP gene were modified in different ways. In one copy (plasmid “A”) the N-terminal NdeI site was removed by mutagenesis (Quikchange), the stop codon was replaced with GCC, encoding alanine and the C-terminal BamHI site was replaced with an NdeI site. In the second copy of the plasmid (plasmid “B”) the N-terminal NdeI site and the stop codon were left in tact while the N-terminal BamHI site was again replaced with NdeI.

Plasmids A and B were both digested with NdeI. In the case of plasmid B, the resulting TRAP gene was removed from the parent plasmid by gel purification (Qiagen) and was added to the linearized plasmid A. Ligation of the TRAP gene from plasmid B into plasmid A was typically carried out using approximately 100 ng of linear plasmid, an excess of insert gene and 175 units of T4 DNA ligase (Takara) in a total volume of 20 μl and incubated for 12 hours at 16° C. The resulting gene construct contained two copies of the TRAP gene connected by the DNA sequence CATATG, the NdeI site encoding amino acids histidine and methionine. This sequence was replaced with the sequence GCCGCCATQ, the final sequence connecting the two copies of the gene being GCCGCCGCCATG translating to amino acids AAAM.

Extending the gene construct with further copies of TRAP was done in a similar way with additional mutagenesis and sequencing reactions to ensure that additional NdeI restriction sites that were not required were removed.

Expression and Purification.

The gene product consisting of three copies of the TRAP monomer (TRAP3) and the product consisting of 4 copies (TRAP4) were expressed in E. coli BL21 DE3 cells (Stratagene). Cell lysate in 50 mM Tris-HCI pH 8.5 was centrifuged at 34,000 rpm, 4° C. for 30 minutes and the supernatant removed. In a typical purification procedure, the supernatant was applied to a Q-sepharose column (Pharmacia) equilibrated in 50 mM Tris HCI pH 8.5 and eluted with an ascending NaCl gradient. TRAP3 was found to pass straight through the column but purified from the majority of contaminating proteins which bound to the column. TRAP4 was found to elute at approximately 200 mM NaCl. TRAP proteins were then applied to a heparin sepharose column (Pharmacia), proteins containing more than 150 mM NaCl were transferred into 50 mM Tris HCI pH 8.5 buffer containing 150 mM NaCI. Proteins were eluted with an ascending NaCl gradient. Both TRAP3 and TRAP4 typically eluted at approximately 200-300 mM NaCl. In cases where further purification of the protein was necessary and to try to remove any bound tryptophan, protein was exchanges into buffer containing 50 mM Tris HCI pH 8.5 and 1 M NaCl and applied to a HiLoad superdex 200 gel filtration column (Pharmacia).

The identity of TRAP3 and TRAP4 were confirmed by MALDI-TOF Mass spectrometry using an Autoflex-YS spectrometer (Brucker).

Electron Microscopy.

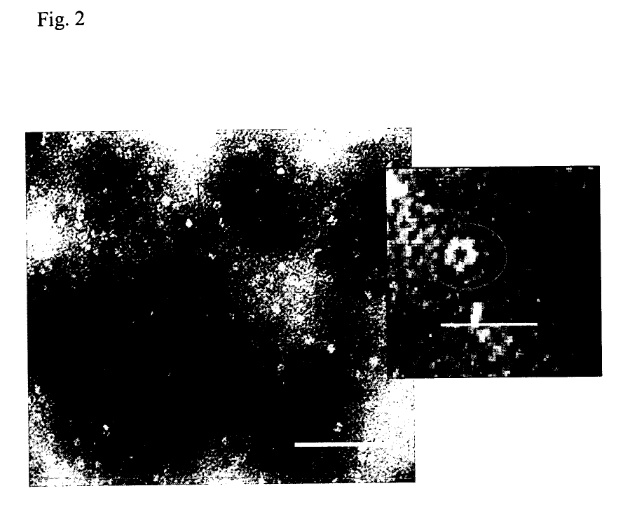

TRAP was further identified using transmission electron microscopy (TEM, FIG. 2). Transmission electron microscopy was carried out using a 200 keV JEM-2200 (JEOL) or a 300 keV JEM-3100 FEF (JEOL). TRAP proteins typically at approximately 1.6 mg/ml in 50 mM Tris pH 8.5, 50 mM NaCl buffer were added to a carbon-coated copper grid and stained with 3% potassium tungsten acetate.

Under TEM observation TRAP rings were clearly visible in the wild type sample and similar rings were also visible in TRAP3 and TRAP4 samples.

Crystallization.

TRAP3 and TRAP4 were crystallized using the hanging drop method. TRAP3 crystals were grown at 20° C. using a stock of approximately 11 mg/ml protein mixed with 10 mM L-tryptophan mixed 1:1 with mother liquor; 30% v/v PEG 300, 90mM CAPS pH 9.5, 150 mM ammonium sulfate. Crystals grew as cubic shaped crystals over a period of 3 weeks. TRAP4 crystals were grown at 20° C. using a stock of approximately 10 mg/ml protein and mixed 1:1 with mother liquor; 40% v/v PEG 200, 90mM CAPS pH 9.5, 200 mM ammonium sulfate. Crystals grew as cubic shaped crystals over a period of 9 days.

Both proteins crystallized in space group P42₁2 with the equivalent of three TRAP monomers per asymmetric unit, with lattice constants: a=110±2 Å, b=110±2 Å, and c=36±2 Å. TRAP3 diffracted to 2.0 Å and TRAP4 to 1.8 Å. Data were collected at beamline BL5 at the Photon Factory, Tsukuba, Japan.

As shown in FIG. 3, the structures of TRAP3 and TRAP4 show almost identical tertiary structure to the wild-type TRAP monomer. The rings formed by TRAP3 and TRAP4 are essentially identical in size and have a central hole 31.1 Å in diameter (between opposing Ser7 Cαs), the height of the ring is 31.8 Å (between Lys40 Cαand Asp29 Cα) and the overall diameter is 81.0 Å (between opposing Glyl 8 Cαs) compared to 27.7 Å, 32.1 Å and 77.0 Å respectively in the wild-type structure.

As shown in FIG. 4, the monomers of TRAP3 and TRAP4 are linked by linking peptide strands.

As shown in FIG. 5, in the crystal structure, the modified TRAP3 and TRAP4 proteins stack in an aligned manner to form an essentially infinite tube (see FIG. 5D). In other structures of TRAP solved, packing does not result in the formation of a continuous tube (see FIG. 5B and 5C).

### INDUSTRIAL APPLICABILITY

The TRAP rings made from TRAP3 or TRAP4 protein have the possibility of being used as biomineralization vessels to biomineralize gold or other metals and/or semiconductors to form arrays of quantum dots (see FIG. 6). The TRAP tubes within crystals may have used as masks for photolithography to produce ultra-thin semiconductor wires or switches. The tubes in the crystal also offer the basis for further modification to produce tubes in solution which could be used to produce as biomineralization vessels to produce nanowires by biomineralizing conductors/semiconductors throughout the length of their cores.

1. A modified TRAP protein formed from a plurality of genetically engineered fusion molecules of TRAP monomers, wherein said fusion molecule consists of two or more wild type TRAP monomer equivalents.

2. The protein of claim 1, wherein said fusion molecule consists of three or four wild type TRAP monomer equivalents.

3. The protein of claim 1, which forms a ring containing the equivalent of 12 wild type TRAP monomers.

4. The protein of claim 1, wherein the wild type TRAP monomers in the fusion molecules are linked by a linking peptide.

5. The protein of claim 1, wherein the gene of said TRAP protein is derived from Bacillus stearothermophilus.

6. A nanotube formed from multiple copies of the modified TRAP protein as defined by claim 1, wherein the modified TRAP proteins stack in an aligned manner to form a tube.

7. A crystal of the protein of claim 2, which has lattice constants: a=110±2 Å, b=110±2 Å, and c=36±2 Å, and is crystallized in space group P42₁2.

8. A crystal of the nanotube of claim 6, wherein the modified TRAP proteins stack in an aligned manner to form close-packed, linear nanotubes.

9. A biomineralization vessel comprising the protein of claim 1.

10. A quantum dot comprising the protein of claim 1 and metal.

11. A nanowire comprising the nanotube of claim 6 and metals.

12. A method for producing a modified TRAP protein formed from a plurality of genetically engineered fusion molecules of TRAP monomers, or a nanotube formed from multiple copies of said modified TRAP protein, which method comprising:
preparing a gene construct comprising multiple TRAP monomer genes;
expressing the gene construct in E. coli cells to produce a genetically engineered fusion molecule of TRAP monomers; and
purifying the expressed fusion molecules.

13. The method of claim 12, wherein the gene of said TRAP protein is derived from Bacillus stearothermophilus.

14. A method for producing ultra-thin semiconductor wires or switches comprising or using the nanotube of claim 6 as masks for photolithography.

15. A biomineralization vessel comprising the nanotube of claim 6.
